(19) United States
(12) Patent Application Publication
(10) Pub. No.: US 2025/0206706 A1
(43) Pub. Date: Jun. 26, 2025

(54) A METHOD OF PRODUCING EXPLOSIVE HMX BY FLOW SYNETHESIS

(71) Applicant: BAE SYSTEMS PLC, London (GB)

(72) Inventors: Matthew Paul Didsbury, USK Monmouthshire (GB); Niall John McWhir, USK Monmouthshire (GB); Daniel Jubb, Aylesbury Buckingham (GB); Stuart Robert Kennedy, Aylesbury Buckingham (GB); Nicola Kennedy, Aylesbury Buckingham (GB); Christopher Jones, Aylesbury Buckingham (GB); Ian Ewart Paterson Murray, Aylesbury Buckingham (GB)

(73) Assignee: BAE SYSTEMS PLC, London (GB)

(21) Appl. No.: 18/847,898

(22) PCT Filed: Mar. 10, 2023
(86) PCT No.: PCT/GB2023/050566

§ 371 Date: Sep. 17, 2024

(30) Foreign Application Priority Data
Mar. 21, 2022 (GB) 2203917.6

(51) Int. Cl. C07D 257/02

(52) U.S. Cl. CPC C07D 257/02

(57) ABSTRACT

The following invention relates to methods of producing explosives from the nitration of TAT by flow synthesis. The invention relates to a method for the flow synthesis manufacture of HMX, (1,3,5,7-tetranitro-1,3,5,7-tetraazacyclooctane), comprising the steps of
i. preparing input flow admixture, comprising TAT (1, 3, 5, 7-tetraacetyl-1, 3, 5, 7-tetrazacyclooctane), P₂O₅, in nitric acid wherein the nitric acid concentration is greater than 95%,
ii. causing the input flow reagent to enter a flow reactor,
iii. heating the reaction chamber in the flow reactor in the range of 60° C. to 80° C., collecting the reacted admixture.

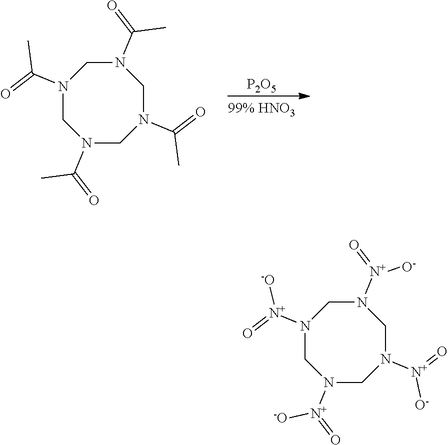

The following invention relates to methods of producing explosives from the nitration of TAT by flow synthesis. Particularly to a method of producing HMX.

Before the present invention is described in further detail, it is to be understood that the invention is not limited to the particular embodiments described, as such may, of course, vary. It is also to be understood that the terminology used herein is for the purpose of describing particular embodiments only, and is not intended to be limiting, since the scope of the present invention will be limited only by the appended claims.

According to a first aspect of the invention there is provided a method for the flow synthesis manufacture of HMX (1,3,5,7-tetranitro-1,3,5,7-tetraazacyclooctane), comprising the steps of
i. preparing input flow admixture, comprising TAT (1, 3, 5, 7-tetraacetyl-1, 3, 5, 7-tetrazacyclooctane), P₂O₅ in excess, in nitric acid wherein the nitric acid concentration is greater than 95%,
ii. causing the input flow admixture to enter a flow reactor,
iii. heating the reaction chamber in the flow reactor in the range of from 60° C. to 80° C.,
iv. collecting the reacted admixture.

After step iv, the reacted admixture may be cooled below 10° C., to cause precipitation of HMX, such as an ice bath.

Preferably, the reacted admixture may be quenched to cause precipitation of HMX. The quench may be caused by mixing the reacted admixture i.e. an output flow, and adding a quenching agent. The quenching agent may be an aqueous solution, such as to cause precipitation of HMX. The quenching agent may be cooled below 10° C.

Highly, preferably in step i) the nitric acid is 99% concentration.

In a further arrangement, before step i) may comprise the steps of
I. preparing input flow reagent A, comprising TAT (1, 3, 5, 7-tetraacetyl-1, 3, 5, 7-tetrazacyclooctane) dissolved in nitric acid with a concentration greater than 95%,
II. preparing input flow reagent B comprising nitric acid greater than 95% concentration and P₂O₅ in excess,
III. causing the input flow reagents A and B to form an admixture and enter the flow reactor.

According to a further aspect there is provided a method for the flow synthesis manufacture of HMX (1,3,5,7-tetranitro-1,3,5,7-tetraazacyclooctane), comprising the steps of
a. preparing input flow reagent A, comprising TAT (1, 3, 5, 7-tetraacetyl-1, 3, 5, 7-tetrazacyclooctane) dissolved in nitric acid with a concentration greater than 95%,
b. preparing input flow reagent B comprising nitric acid with a concentration greater than 95% and P₂O₅ in excess,
c. causing the input flow reagents A and B to form an admixture and enter the flow reactor,
d. heating the reaction chamber in the flow reactor in the range of from 60° C. to 80° C.

After step III and d, the reacted admixture may be cooled below 10° C., to cause precipitation of HMX, such as an ice bath.

Preferably, the reacted admixture may be quenched to cause precipitation of HMX. The quench may be caused by mixing the reacted admixture i.e. an output flow, and adding a quenching agent. The quenching agent may be an aqueous solution, such as to cause precipitation of HMX. The quenching agent may be cooled below 10° C.

The use of flow synthesis provides a facile means of preparing HMX at both laboratory R&D scale of ˜100 g, and to provide the ability to add further flow reactors to readily scale up production, without the associated dangers of forming +100 kg of HMX explosive in a single reactor vessel. Further, it also avoids the use of hundreds of litres of highly concentrated acid in a large reactor vessel in a batch process. The use of flow synthesis allows for the continuous removal and safe stowage of final explosive product material from the flow reactor or flow reactors, to avoid the build-up of large quantities of explosive material. This may allow explosive processing buildings to process a greater mass of explosive and/or associated safety distances to be reduced, as the explosive material may be distributed to safe areas, away from the flow reactor, as it is synthesised.

The TAT may be added to the input flow reagent A nitric acid in any wt % up to and including a near saturated solution. The higher the concentration of TAT in the input flow reagent A, the more efficient the process. It is highly preferable to dissolve the TAT in the nitric acid, as short a time as possible before flowing into the reactor, to reduce the likelihood of the nitration reaction starting.

The TAT may be dissolved in nitric acid with a concentration in the range of from 95% to 99%, the use of other solvents to aid dissolving the hexamine, may be added.

The TAT:P₂O₅ has a molar excess of P₂O₅, preferably greater than a factor of two molar excess, preferably in the range of from 2 to 30 molar excess, it may be higher.

Preferably input flow reagent A contains only TAT and nitric acid with a concentration in the range of less and 92%.

To assist in achieving the desirable concentration of nitric acid to start nitration of TAT, the input flow reagents A and B may be premixed in a mixing chamber before entering the flow reactor.

The total nitric acid concentration when input flow reagent A and input flow reagent B contain only nitric acid as the acid and the sole nitration agent, the concentration must be sufficient for nitration to occur, such as for example greater than 95% concentration.

The flow rate of input flow reagent A may be selected from any suitable flow rate with input flow reagent B, to provide a total nitric acid concentration capable of causing nitration of TAT, such as for example in the range of greater than 95%. The actual flow rate of input flow reagent A may be microlitres per minute through to millilitres to litres per minute, depending on the capacity of the flow cell.

The flow rate of input flow reagent B may be selected from any suitable flow rate with input flow reagent A to provide a total nitric acid concentration capable of causing nitration of TAT, such as for example in the range of greater than 95% concentration. The actual flow rate of input flow reagent A may be microlitres per minute through to millilitres to litres per minute, depending on the capacity of the flow cell.

The use of other strong acids, such as for example oleum, may be used.

The temperature in the flow reactor needs to be controlled to prevent a highly exothermic reaction from occurring, but providing sufficient heat to sustain the reaction. The step iii) of the reaction chamber is in the range of from 60° C. to 80° C., highly preferably in the range of from 70° C. to 75° C. The temperature is controlled by water circulators. The flow reactor may be heated and/or cooled by any suitable means such as for example water circulator or electric heaters/coolers.

The HMX precipitate is filtered and collected and then washed in a quenching solution. The quenching solution is preferably aqueous, and preferably pH 7 or less.

According to a further aspect of the invention, there is provided the use of flow synthesis for providing explosives from TAT.

According to a further aspect of the invention, there is provided apparatus for carrying out the process according to any one of the preceding claims, wherein 10 the apparatus is modified for explosive compatibility.

### Experimental Reagents

99% HNO3 was purchased from Honeywell in a 500 mL quantity. Cat. 84392-500ML, Lot. No. 1345S.
70% HNO3 was purchased from Fisher scientific in a 2.5 L quantity. Code: 15 N/2300/PB17. Lot: 1716505.
Hexamine was purchased from Sigma-Aldrich in a 250 g quantity. Cat. 797979-250G, Lot. No. MKCJ7669.
Oleum was purchased from Fisher in a 500 mL quantity. Cat. S/9440/PB08, Lot. No. 1689177.

### EXPERIMENTAL

### HMX Example

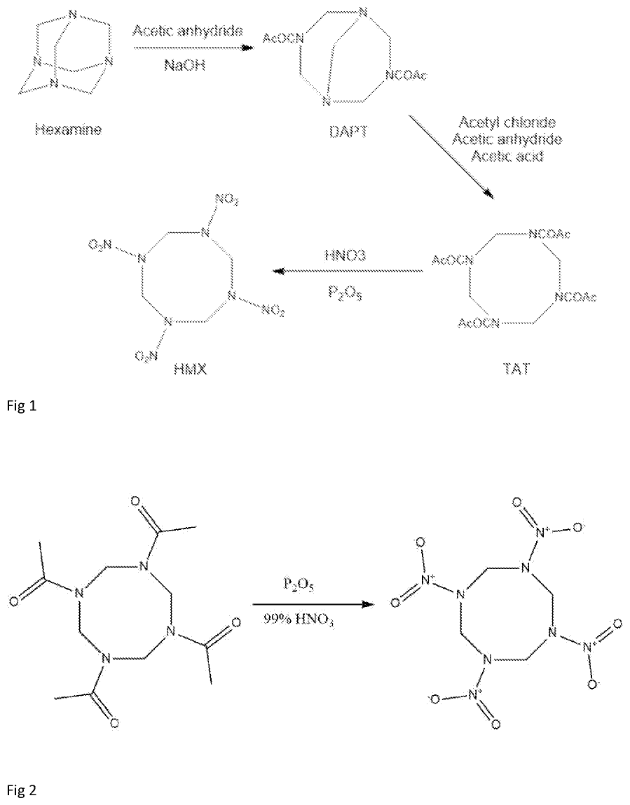

TAT, as shown in FIG. 1, can be readily synthesised from hexamine via DAPT as an intermediate. The main advantage of going through this route is that an 8-membered ring is formed therefore eliminating the possibility of forming RDX as a by-product.

The synthesis of DAPT and TAT are typically high-yielding and are explosive precursors, and so are not explosive, that is they do not sustain detonation. Therefore, these explosive precursors can be made safely in bulk via a batch process, if preferred. Therefore, only the conversion of TAT to HMX needs to be undertaken by flow chemistry to mitigate the build-up of large mass of explosive products, compared to existing traditional batch processes.

The synthesis of DAPT is straightforward and the resulting product can be easily recrystallized from acetone to produce large pure crystals. The ¹H NMR of DAPT was confirmed from by comparing it to literature results.

The subsequent synthesis of TAT is again straightforward and can be carried out on a large scale. The product was recrystallised into a white powder by sonicating the sample in a small amount of acetone. The product purity was confirmed by ¹H NMR. The spectrum contains two major peaks with a 12:8 integration ratio, corresponding to the 4×CH₃ and the 4×CH₂ protons, respectively.

The TAT can be directly converted to HMX, as shown in FIG. 2, using nitration in a flow synthesis arrangement to control the rate of production of explosive material.

### Experimental 1

Line A—a solution of 100 mg TAT, 1000 mg P₂O₅ and 2 mL of 99% HNO₃ were premixed in a single solution and passed through a Labtrix reactor. The preferred reaction time of 120 seconds through the flow synthesis reactor provided sufficient time for nitration to occur. The flow synthesis was performed at a temperature greater than room temperature, it was found that a temperature of 75° C. provided a temperature which allowed the reaction to proceed, but not sufficient to cause an unwanted explosive event. HMX was isolated, without any RDX contamination being present.

### Experiment 2—Scaled Up Protrix Reactor

Line A: 2.0061 g TAT and 20.0357 g of P₂O₅ were dissolved in 40 mL 99% HNO₃
Line B was used as an emergency flush and was primed with 70% HNO₃.

Line A and the Protrix were initially primed with 70% HNO₃ followed by 99% HNO₃. The reaction mixture was prepared in stages. Initially P₂O₅ is slowly dissolved into a stirred solution of 99% HNO₃. This solution was kept in an ice bath. This resulted in an opaque-yellow solution. The addition of TAT to this solution reduced the opacity of the solution however, the reaction mix remained opaque.

Line A was then primed with the reaction mixture.

TABLE 1

| | | | Flow A | P | |
|---|---|---|---|---|---|
| EXP | Temp° C. | Time (s) | (mL) | (bar) | Observation |
| 0168 | 75° C. | 120 | 1.66 | 1.1 | Collected for 12 |
| | | | | | minutes into ice |

The solution, after passing through the Protrix, was left overnight, which resulted in the formation of crystals. These were isolated, washed with water followed by acetone, and then analysed using NMR spectroscopy. The ¹H NMR spectrum of experiment 0168 shows that there are multiple species present in the sample. Some of these peaks correspond to unreacted TAT and partially nitrated TAT.

Highly preferred equivalents of reactants by weight are 1:10:30 of TAT:P₂O₅:HNO₃. The ratio of nitric acid may be further increased in the range of from 20 to 60. The TAT:P₂O₅ has a molar excess of P₂O₅, preferably in the range of from 5 to 30 molar excess. The nitric acid may be present in even higher amounts, however it is preferred to keep optimal mounts to reduce wastage and handling of excess acids.

1. A method for the flow synthesis manufacture of HMX (1,3,5,7-tetranitro-1,3,5,7-tetraazacyclooctane), the method comprising:
preparing input flow admixture, comprising TAT (1, 3, 5, 7-tetraacetyl-1, 3, 5, 7-tetrazacyclooctane), P₂O₅ in excess, in nitric acid, wherein the nitric acid concentration is greater than 95%;
causing the input flow reagent to enter a flow reactor having a reaction chamber;
heating the reaction chamber in the flow reactor in the range of 60° C. to 80° C.; and
collecting the reacted admixture.

2. The method according to claim 1, wherein after collecting the reacted admixture, the method comprises causing the reacted admixture to cool to cause precipitation of HMX.

3. The method according to claim 1, wherein the nitric acid is 99% concentration.

4. The method according to claim 1, wherein heating the reaction chamber includes heating the reaction chamber in the range of 70° C. to 75° C.

5. The method according to claim 1, wherein collecting the reacted admixture includes mixing a quenching agent and an output flow of the flow reactor.

6. The method according to claim 1, comprising quenching the collected reacted admixture to cause precipitation of HMX.

7. The method according to claim 6, wherein the quenching includes use of a quenching agent that is an aqueous solution, to cause precipitation of HMX.

8. The method according to claim 6, wherein the quenching includes use of a quenching agent that is cooled below 10° C.

9. The method according to claim 1, wherein the reactants are 1:10:30 of TAT:P₂O₅:HNO₃.

10. The method according to claim 1, wherein prior to preparing the input flow admixture, the method includes:
preparing input flow reagent A, comprising TAT (1, 3, 5, 7-tetraacetyl-1, 3, 5, 7-tetrazacyclooctane) dissolved in nitric acid with a concentration greater than 95%;
preparing input flow reagent B comprising greater than 95% concentration nitric acid and P₂O₅; and
causing the input flow reagents A and B to enter the flow reactor.
